(19) United States
(12) Patent Application Publication
(10) Pub. No.: US 2018/0265885 A1
(43) Pub. Date: Sep. 20, 2018

(54) HIGHLY EXPRESSED PROTECTIVE PLANT-DERIVED BROADLY NEUTRALIZING HIV MONOCLONAL ANTIBODIES FOR USE IN PASSIVE IMMUNOTHERAPY

(71) Applicant: Plantvax, Inc., Rockville, MD (US)

(72) Inventor: Yvonne Rosenberg, Rockville, MD (US)

(73) Assignee: Plantvax, Inc., Rockville, MD (US)

(21) Appl. No.: 15/547,787

(22) PCT Filed: Jan. 29, 2016
(86) PCT No.: PCT/US2016/015813

§ 371 Date: Jul. 31, 2017

Related U.S. Application Data
(60) Provisional application No. 62/109,225, filed on Jan. 29, 2015.

(51) Int. Cl. C12N 15/82; A61K 39/42; C07K 16/08; C07K 1/14; G01N 33/68; A61P 31/18

(52) U.S. Cl. CPC C12N 15/8258; A61K 39/42; C07K 16/08; A61P 31/18; C07K 1/14; G01N 33/6854; C12N 15/8205

(57) ABSTRACT

Monoclonal antibodies produced in plants at high levels wherein elimination of L chain glycans improves plasma stability of certain plant derived bnAbs are provided. Monoclonal antibodies produced in plants which exhibit no or reduced immunogenicity after multiple injections are provided. Methods of production of the foregoing are provided. Methods of determining immunogenicity assessing the immunogenicity and neutralizing ability of the same are provided. Methods of treating HIV using monoclonal antibodies produced in plants are also provided.

### BACKGROUND

Although early studies demonstrated that passive transfer of HIV broadly neutralizing monoclonal antibody (bnAb) cocktails protected macaques against challenge with chimeric simian-HIV (SHIV) isolates, very high doses were required because the prototypic HIV mAbs possessed only moderate neutralizing activity. Recent developments in large scale screening of HIV+ individuals known to have broadly neutralizing HIV antibodies in their circulation, together with efficient single cell antibody cloning techniques, have led to the identification of increasingly potent HIV bnAbs. These advances, and the finding that antibodies best correlate with protection in clinical vaccine trials, have greatly increased the prospect that therapeutic strategies involving passive immunotherapy will find application in preventing infection in the case of mother-to-child transmission and sexual transmission and in controlling both acute and chronic infections.

HIV envelope epitopes, which form the targets of these potent and broadly neutralizing antibodies, generally fall into several categories; those predominantly targeting either the CD4 binding site (CD4bs) epitopes or epitopes partly comprising carbohydrates on the gp120. More recently, potent broadly neutralizing mAbs specific for the membrane proximal external region (MPER) e.g. 10E8 or for an epitope spanning both gp120 and gp41 e.g. 35022, 8ANC195, have also been identified. Within the family of glycan epitopes, subgroups are becoming evident, although almost all mAbs are directed towards oligomannose glycans e.g. (i) the high mannose epitopes on the V1/V2 variable loop (PG9/PG16) and (ii) the N332A sensitive complex glycan on the V3 loop (2G12, PGTs, 10-1074). In the latter group, however, minor differences may lead to marked changes in potency. Thus, while PGT128 interacts with two oligmannose glycans N301 and N332 as well as with the base of the V3 loop, the more potent PGT121 mAb appears more dependent on N332 than N301 and uniquely recognizes a complex glycan epitope terminating in galactose or α2-6-linked sialic acid.

Furthermore, the identification of highly potent broadly neutralizing antibodies (bnAbs) against HIV-1, and success in preventing SHIV infection following their passive administration, have increased the likelihood that immunotherapeutic strategies can be adopted to prevent and treat HIV-1 infection. While broad and potent in vitro neutralization potency (IC50) is a prerequisite for a mAb's ability to passively protect against or control HIV in vivo, its therapeutic potential will be influenced by its in vivo properties e.g. plasma stability and immunogenicity as well as ease and cost of production. Antibodies against therapeutics are commonly elicited and the clinical implications e.g. drug neutralization, are becoming better understood. In the context of passive mAb treatment, the development of anti-drug antibodies e.g. adalimumab, has been associated with lower mAb concentration and loss of efficacy of the drug. This challenge, in addition to the rapid emergence of viral escape mutants in infected recipients will likely necessitate constant development of new potent antibody-based therapies on an on-going basis to counteract both viral resistance and the potential spread of a certain HIV-1 clade.

Based on the foregoing, what is needed is an expression system that provides speed, versatility, a pathogen-free nature and low-tech requirements, in particular in the early developmental stages from cloning to preclinical protection studies.

### SUMMARY OF THE INVENTION

In general, the foregoing and other objects are achieved with the invention as follows:

In one aspect, plant-based transient expression systems are provided which offer unique advantages in their speed, versatility, pathogen-free nature and low-tech requirements, in particular in the early developmental stages from “cloning to preclinical protection studies”.

More particularly, in an aspect of the present invention, the transient plant N.b/p19 system is used to produce and test different glycoforms of the bnAbs VRC01, PG9, PG16, 10-1074, NIH45-46^{G54W}, 10E8, PGT121, PGT128, PGT145, PGT135 in addition to b12 and mutated forms of VRC01 (mVRC01) and NIH45-46^{G54W} (mNIH45-46^{G54W}) and to assess their in vivo properties such as plasma stability, immunogenicity and efficacy of protection in macaques to distinguish those most likely to comprise or become a component of an affordable and efficacious immunotherapeutic, both singly or as a cocktail.

In further aspects of the present invention, in addition to HIV bnAbs, production of conventional anti-HIV antibodies which are less mutated, exhibit lower potency and breadth of neutralizing activity and high FcR-mediated activity e.g. antibody-dependent cell cytotoxicity (ADCC) and antibody-dependent cell-mediated viral inhibition (ADCVI) similar to those generated by current HIV vaccines, can also successfully be produced in plants.

In yet further aspects, plasma stable, non-immunogenic, and broadly neutralizing HIV monoclonal antibodies are provided as are methods of making the same and assessing the immunogenicity of the same.

In another aspect, a method of treatment is provided wherein both pregnant women and their new borns receive bnAbs, preferably via non-invasive routes e.g. orally, or alternatively where the mother receives the antibody via intravenous injection and the new born via subcutaneous injection or non-invasively.

### BRIEF DESCRIPTION OF THE DRAWINGS

FIG. 1 shows very high expression levels of PGT121 production in plants. Samples of N.benthamiana leaf extracts were serially two-fold diluted so samples represented 2,000-31 ug of leaf biomass (left panel) and compared with known concentrations (100-800 ng) of purified plant-derived PGT121 (right panel). The intensity of staining indicated that ˜125 ug of leaf biomass (*) was equivalent to ˜200 ng of protein(*), conservatively being the equivalent of 1.3-1.6 g/kg of leaf biomass.

FIG. 2 shows neutralization titers of N.benthamiana-derived HIV bnAbs and a control CHO-derived bnAb cocktail against Tier 2 HIV isolates (A) and against SHIV isolates (B) using pseudovirus-based TZM-bl cells. Leaves were harvested at days 10-12 following Agrobacterium infiltration and leaf extracts were purified using a Protein A column; producing 100-1,300 mg/kg of purified bnAb. All bnAbs shared the same constant region of kappa light and gamma-1 heavy chain. IC50s represent the concentration at which relative luminescence units (RLUs) are reduced by 50% versus virus control wells. IC50 values are color coded as indicated. Control bnAb produced in mammalian cell e.g. CHO-derived PGT128, HEK-293-derived VRC01 and a pool of two CHO-1 (PG9-like) and CHO-31 (VRC01-like) bnAbs which strongly neutralizes all HIV isolates. The most potent bnAb PGT121 is highlighted in dashed lines.

FIGS. 3A and 3B shows neutralization titers of N.benthamiana-derived bnAb b12 compared to a control CHO-derived b12 against 8 HIV and SHIV isolates using pseudovirus-based TZM-bl cells. Leaves were harvested at days 10-12 following Agrobacterium infiltration and leaf extracts were purified as described.

FIG. 4 Shows the binding of purified b12 glycoforms, harvested at different times post infiltration, with rsCD64(FcgRI) at 25° C. using protein-A captured b12 antibody preparations and HBS-EP as running buffer. Binding curves are double referenced and have been normalized to equal capture levels. Thus, the signals are directly comparable and reflect genuine difference in binding site activity and affinity.

FIG. 5 shows the glycosylation profile of non-KDEL and KDEL glycoforms of plant-derived bnAbs. MALDI TOF analysis was performed on the non-KDEL glycoforms of b12 and VRC01 and the ER retained KDEL-b12, KDEL-VRC01, KDEL-2G12 and KDEL-PG9 glycoproteins to determine the percentages of glycans present on each.

FIGS. 6A, 6B and 6C show circulatory clearance of plant-derived bnAbs in macaques at different times following intravenous (IV) injections as measured using ELISA. (FIG. 6A) Average ug/kg of two macaques injected with b12 (5 and 7.5 mg/kg), 10E8 (5 mg/kg), 10-1074 (5 mg/kg) were assayed using plant-derived HIV env-coated plates while PGT121 (5 mg/kg) was measured using HIV Bal gp120 env-coated plates. (FIG. 6B). Comparison of clearance of 5 mg/kg of 10-1074 and mNIH45-46 G54W in a separate study (OD450 units) (FIG. 6B). Example of more rapid circulatory clearance of plant-derived PGT121 in macaques. Close association between ug/ml as measured by ELISA and ID50 against the RHPA4259.7 isolate as measured by pseudovirus-based TZM-bl assay at different days following IV injection of 5 mg/kg into each of two macaques (FIG. 6C).

FIGS. 7A and 7B show circulatory clearance of plant-derived in macaques at different times following i.v. injections of plant-derived VRC01 and mVRC01 as measured using ELISA (FIG. 7A) or using both ELISA and pseudovirus-based TZM-bl neutralization assay (FIG. 7B). Data are an average of two macaques. (A) ug/ml levels following injection of mVRC01 at 5 and 10 mg/kg and VRC01 (10 mg/kg). The rapid clearance of non-mutated VRC01 (10 mg/kg) in two macaques is shown in the insert. (B) Comparison of the plasma samples from the macaques receiving 10 mg/kg by both ELISA (ug/ml) and neutralization (ID50) against the HIV Tier 2 RHPA4259.7 isolate using TZM-bl assay.

FIG. 8 shows the circulatory clearance profiles of 5 mg/kg of plant-derived PGT121 in African Green monkeys following subcutaneous (SC)(#8291, 8338) and intramuscular (IM)(8288,8390) injections. ID50s were assessed at different times after injection using a pseudovirus-based TZM-bl assay against the Tier 2 SHIV-SF162P3. The protective ID50 (˜100) is indicated.

FIGS. 9A and 9B show immunogenicity of plant-derived bnAbs following a second I.V. injection of antibody in macaques as measured by anti-bnAb production. (A) Responses against b12 and VRC01 in macaques following two injections two weeks apart. WT VRC01 (4.5 mg/kg and 10 mg/kg) was injected into macaques (#5191, 5193) and b12 (4.5 mg/kg and 7.5 mg/kg) was injected into macaques (#5194, 5192). Plasma samples collected from each of the macaques at the time indicated were then assayed by ELISA against both VRC01 (anti-VRC01) and b12 (anti-b12) to demonstrate anti-bnAb levels. (B) Immunogenicity of plant-derived VRC01, 10-1074 and NIH45-16 in macaques following two injections of 5 mg/kg two weeks apart (A) Macaques #5544, 5545 received two injections of mVRC01, #5540, 5542 received 10-1074 and 5541,5543 received mNIH45-46G54W. Plasma samples collected from each of the macaques at the time indicated were then assayed by ELISA against the bnAbs they had received (anti-VRC01, anti-10-1074 and anti-mNIH45-46G54W) to demonstrate anti-bnAb levels. All were tested against b12 as a negative control.

FIG. 10 shows the comparison of plasma levels of anti-VRC01 antibody in all macaques injected twice I.V. with VRC01 (numbers indicated) by ELISA using plant-derived VRC01 (solid fill) (10A) and HEK-203-derived VRC01 (hatched fill) (10B). Times of the bleeds are indicated.

FIG. 11 shows the reactivity of plasma from macaques injected with different bnAbs or naïve macaques. (A) Plasma samples from the 11 injected macaques were collected 2-3 weeks following the second injection and idiotype specificity was demonstrated by ELISA against PGT121, 10-1074, VRC01 and NIH45-46 produced in mammalian cells. (B) Screening of 14 naïve macaques for pre-existing reactivity against PGT121, 10-1074, VRC01 and NIH45-46G54W (produced in mammalian cells) prior to injection.

FIG. 12 shows idiotypic-specific Inhibition of bnAb neutralization by macaque anti-idiotypic antibody against HIV RHPA4259.7 and SHIV-BaL-P4.

FIGS. 13A, 13B and 13C show the protection against SHIV BalP4 challenge by a cocktail of plant-derived bnAbs in macaques as measured by log 10 rRNA (copies/ml of plasma). (A) A cocktail of plant-derived VRC01, 10-1074, b12 and 10E8 (5 mg/kg each) was administered intravenously 6 hours prior to intravaginal challenge with SHIV Bal P4 with all four macaques (#5735, 5736, 5737, 5750) protected with VL<2 logs at every time point indicated or (B) A cocktail given 6 hours post challenge with 2/4 macaques (#5736 and 5737) protected (VL<2 logs). The log 10 vRNA (copies/ml of plasma) of three untreated infected control macaques receiving no bnAb are shown in FIG. 13C.

FIG. 14 shows protection by plant-derived PGT121 administered subcutaneously against intravaginal SHIVSF162P3 challenge in rhesus macaques as measured by log 10 rRNA (copies/ml of plasma). Macaques were bled and assayed at the time points indicated. (A) Protection in 6/6 macaques (#12N010, 11N006, 13N010, 04N013, JFL, 07N008) that received 3.7-7.1 mg/kg doses of PGT121 given 24 hr prior to SHIV SF162P3 challenge (VL<2 logs). Insert shows a linear correlation between ID50 and dose (3.5-7.1 mg/kg) at 24 hr; ID50 values at doses of 3.86, 4.7 and 5.6 mg/kg were 1:202, 1:612 and 1:738 respectively). (B) Protection in 5/6 macaques administered 5 mg/kg SC immediately after SF162P3 challenge (VL<2 logs) with one macaque (#13N010) with VL>5 logs. (C) Protection in 3/4 macaques that received 1 mg/kg PGT121 6 hr prior to challenge. (D) Log 10 vRNA (copies/ml of plasma) four control macaques receiving no passive bnAb.

### DETAILED DESCRIPTION OF THE INVENTION

The present invention is directed, inter alia, to provision of plant-derived neutralizing HIV monoclonal antibodies. When used as immunotherapeutics, these plant-derived neutralizing HIV monoclonal antibodies are broadly neutralizing (bnAbs) and protective. The plant-derived neutralizing HIV monoclonal antibodies are also highly expressed when produced in plants.

### Production of HIV MAbs in Plants

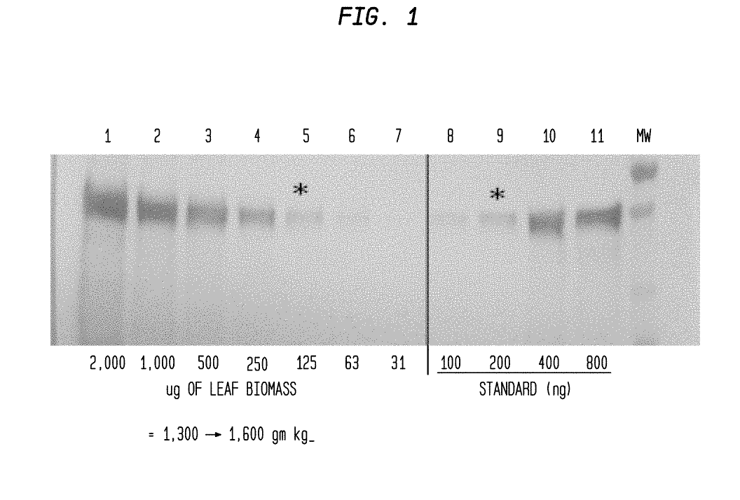

KDEL and non-KDEL glycoforms of HIV bnAbs have been transiently produced in both Nicotiana tabacum (harvested <D7) and N.benthamiana (harvested D6-14); each sharing the same kappa light (CL) and the IgG1 heavy (CH) chains; the latter containing a glycan at N297 in the CH2 domain and some containing VL glycans. Using a p19/N.benthamiana (p19/N.b) transient system, monoclonal antibodies are produced from 100-1,600 mg/kg (Example 1, FIG. 1).

Neutralization Activity of Plant-Derived bnAbs

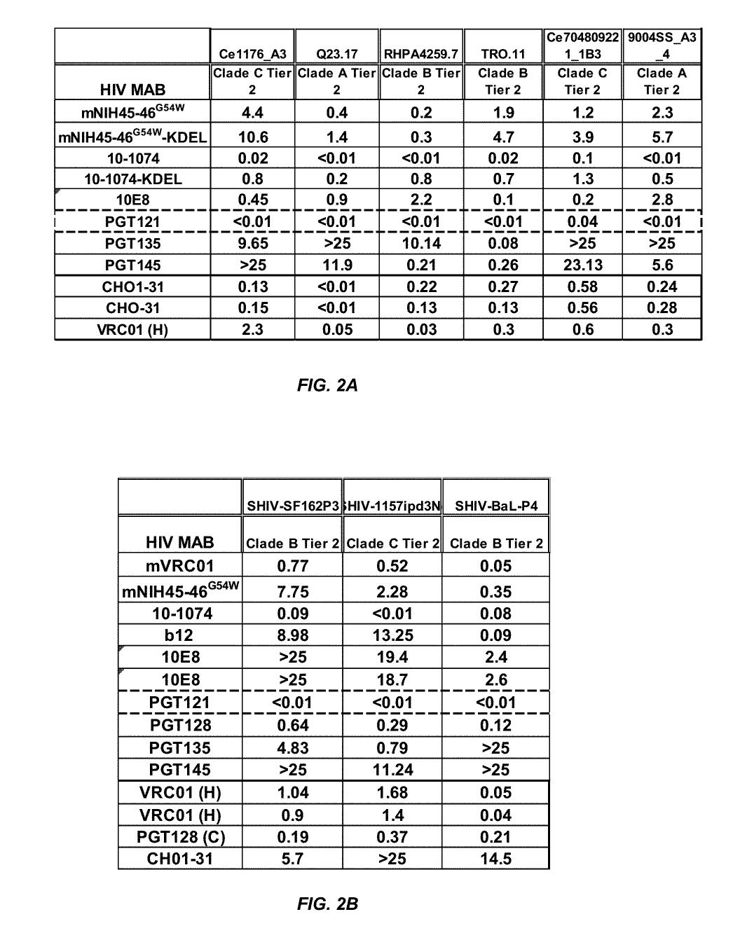

Referring now to FIGS. 2A and 2B, neutralization activity of KDEL-tagged (usually high mannose) and non-KDEL-tagged (usually complex) glycoforms of N.b/p19-derived mAb and mammalian cell controls were measured in a TZM-bl assay as a reduction in Luc reporter gene expression after a single round of infection with a small panel of Env-pseudotyped viruses, including six tier 2 HIV-1 isolates (FIG. 2A) and three simian/human HIV (SHIV) isolates (FIG. 2B). Overall, with the exception of NIH45-46G54W, all plant-derived bnAb IC50s were similar to controls; PGT 121 exhibiting the highest potency with an IC50 of <0.01 against 8/9 HIV and SHIV isolates. Surprisingly, the high mannose glycoforms, 10-1074-KDEL and NIH45-46^{G54W} and NIH45-46^{G54W}-KDEL had lower neutralization activity than the non-KDEL complex glycoforms. Potency against SHIV isolates SF162P3, 1157ipd3N4 and SHIV-Bal-P4, was assessed to determine the most appropriate challenge isolate for macaque passive protection studies. 10-1074 and PGT121 had the lowest IC50 against the clade C 1157ipd3N4.

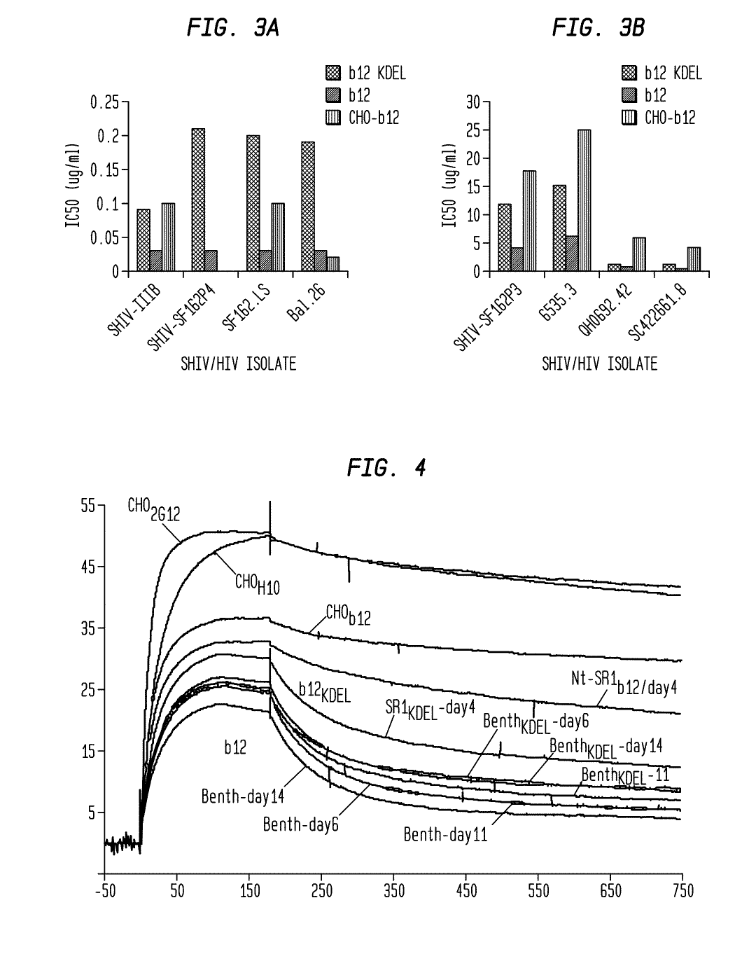

Most high mannose and secreted complex glycoforms of the HIV mAbs were produced transiently in N. tabacum and N. benthamiana and usually exhibited equivalent neutralizing activity (IC50s) as compared to their counterparts produced in mammalian cell cultures, except notably plant-derived b12 which had IC50s lower than CHO-derived against several isolates (FIGS. 3A and 3B). Methods for improving yields of purified bnAbs are used such that Protein A-purified PGT121 reaching levels 1,300 mg/kg plant leaf biomass and Protein A-purified VRC01 reaches levels of 300-600 mg/kg of plant leaf biomass. An analysis of the ability of purified plant-derived b12, harvested at different times after infiltration, to bind to HIV gp120 env was performed (FIG. 4) and how N-linked glycosylation profiles could account for differences in HIV env binding.

Trafficking/Deposition of bnAbs within the Plant Host Cell and Effects on Post-Translational Modification e.g. Glycosylation (Example 4).

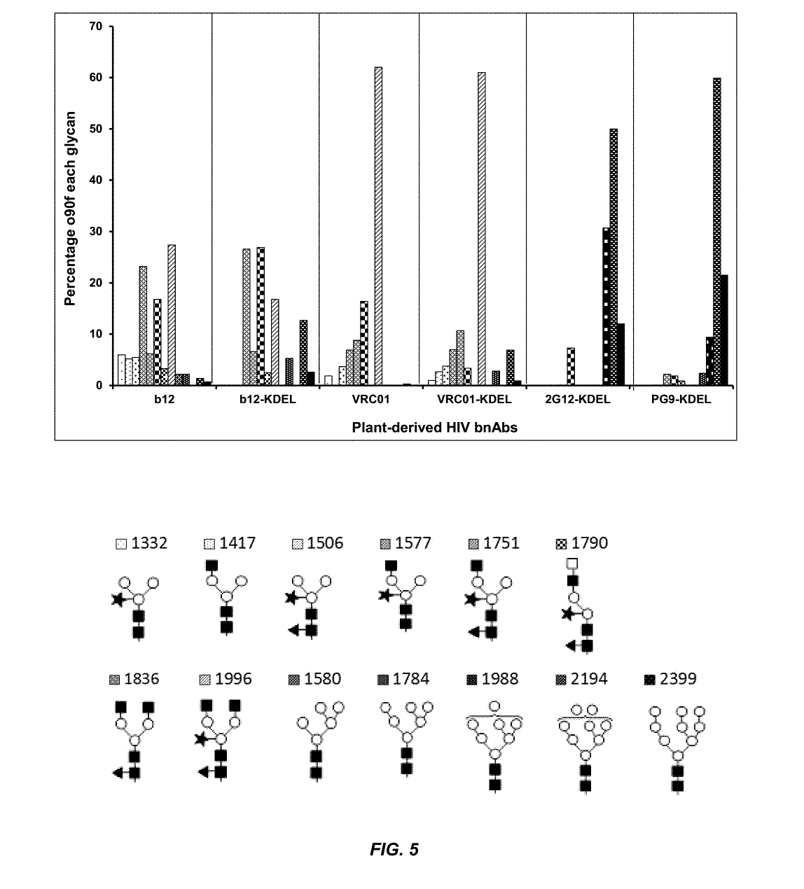

Referring now to FIG. 5, in order to determine a role for N-linked glycosylation in the different binding properties of certain bnAbs to HIV env and FcR, the synthesis, targeted trafficking and deposition of plant-derived non-KDEL and KDEL-linked b12 and VRC01 b12 into different plant cell compartments was investigated with KDEL-2G12 and KDEL-PG9 controls.

The results provide new insights into the synthesis, targeted trafficking and deposition of these mAbs into different plant cell compartments. More importantly, since all mAbs tested share an identical Fc region, glycan analysis suggests that, unlike other KDEL-tagged proteins, specific peptide sequences in the b12 and VRC01 Fab regions are able (i) to signal translocation of KDEL-tagged H and L chains from the membranes of the ER, (ii) to then unexpectedly route trafficking via the golgi complex and (iii) to subsequently signal entry into the lytic environment of the vacuoles. These data show that targeting sequences on b12 and VRC01 mAbs override KDEL retention signals and permit/allow further Golgi-dependent trafficking to the vacuoles with appended compartment-specific glycans, including the novel glycan at theoretical mass 1790 (HexNAc1GlcNAc1Xyl1Man2GlcNAc2Fuc1)(Example 4).

In Vivo Properties of Highly Potent Plant-Derived HIV bnAbs

While high in vitro neutralization potency is a prerequisite for an antibody's ability to passively protect against or control pathogens in vivo, its therapeutic potential will also depend on its plasma stability and immunogenicity, as well as ease and cost of production. In the case of HIV, it is now well established that the breadth and potency of bn mAbs correlate closely with their level of somatic mutation and the frequency of indels (insertions/deletions). Since the efficacy of any immunotherapeutic clearly depends on circulatory stability, it was important to determine whether certain distinctive properties of the bnAbs e.g. high rates of H and L chain mutations (˜17-32%), long heavy chain complementarity-determining region 3 (HCDR3) and plant-specific glycosylation, significantly impact their plasma retention time as well as their immunogenicity and immune regulation following in vivo administration.

### (I) Pharmacokinetics

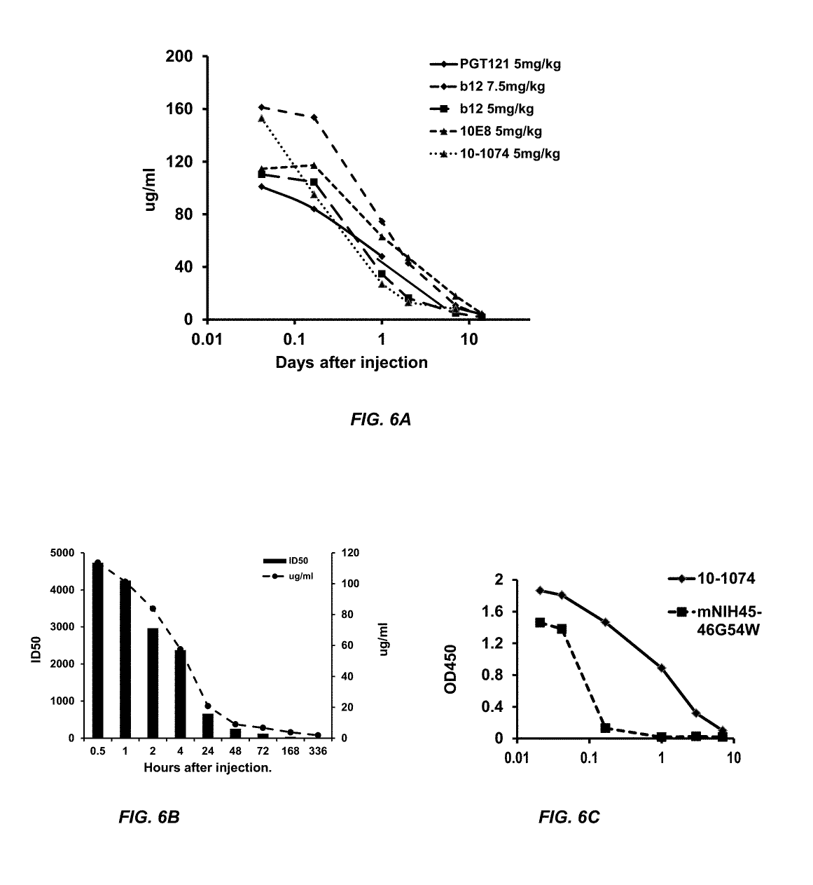

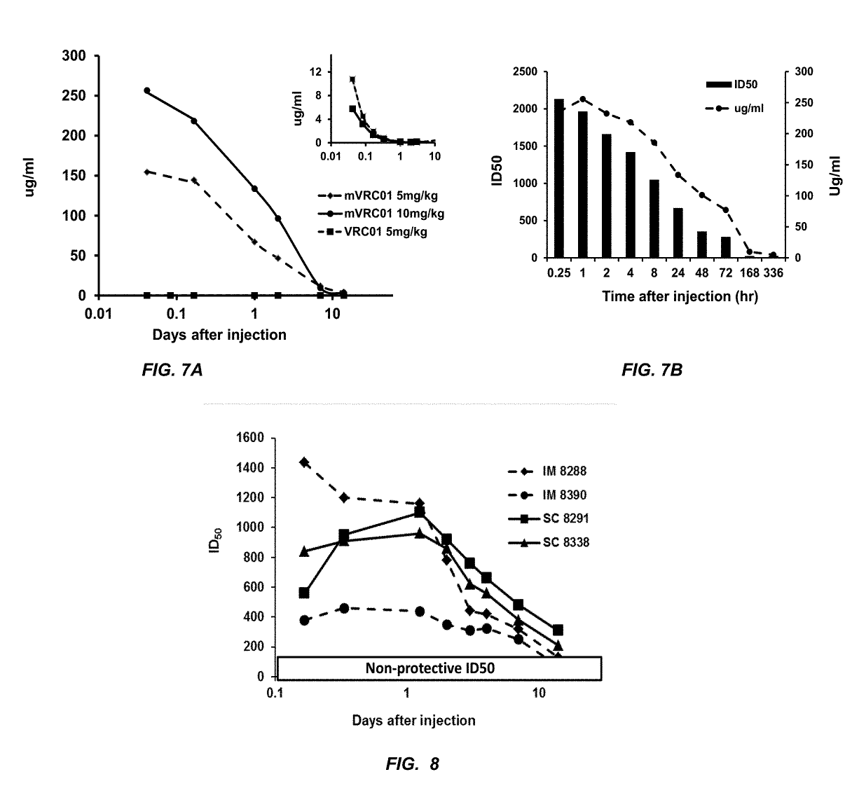

To assess the clearance rates of each of the plant-derived bnAbs and their potential for in vivo use, plant-derived bnAbs were injected I.V. at different doses (5-10 mg/kg) into macaques to assess in vivo stability. Circulating levels of each bnAb were assessed in sera or plasma of the injected macaques either (i) by ELISA using wells coated with either plant-derived high mannose or complex HIV envelope glycoforms or (ii) by measuring the ID50 of the macaque plasma or serum in a TMZ-bl neutralizing assay; both being closely correlated. See Example 5 with reference to FIGS. 6 and 7.

Most of the plant-derived bnAbs exhibited similar pharmacokinetic profiles. In two related antibodies however, plasma clearance was unexpectedly rapid. Since all of the plant-derived bnAbs shared the same CH and CL domains, the rate of clearance may depend on specific locations of carbohydrates as well as and the host specific glycosylation pattern, these plant-derived proteins were mutated to eliminate any VH and VL glycans and re-assessed for any effects on plasma retention. (Example 6 and referenced in FIGS. 6 and 7). Interestingly, these mutations did not normalize retention of one of the bnAbs (NIH45-46) (FIG. 6B) but did normalize the rate of clearance of another (VRC01)(FIG. 7A).

Parenteral injections include those delivered intravenously (IV), intramuscularly (IM) and subcutaneously (SC). Intravenous routes of delivery, especially in the case where multiple administrations are required and in locations are not easily accessible e.g. people in rural areas of Africa, are not practical and I.M. injections can have variable results. This is especially relevant for babies where neither IV nor IM delivery of treatments are appropriate. For this reason, pharmacokinetics following SC and IM delivery were compared to the proof-of-concept results achieved initially by IV injection in monkeys and the rate of clearance compared (Example 7 and referenced in FIG. 8).

(ii) Immunogenicity of Human HIV bnAbs

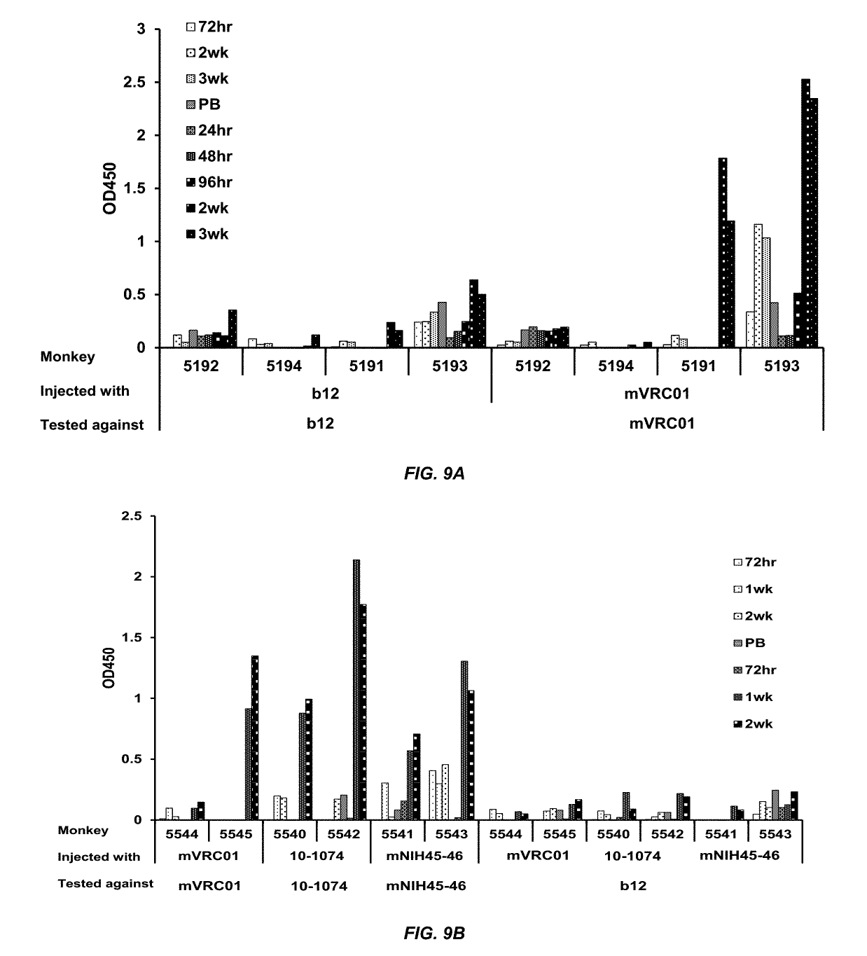

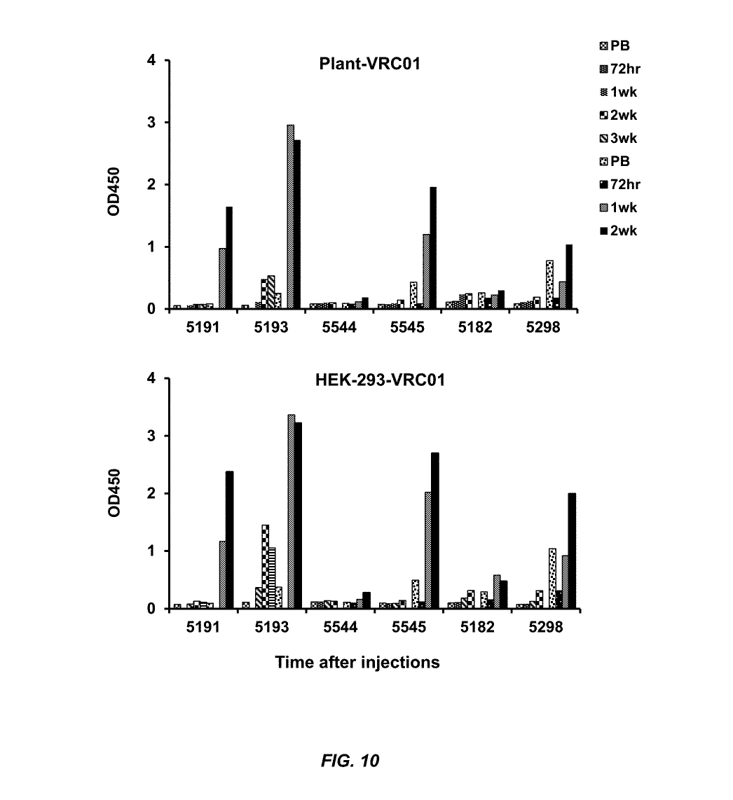

Since passive immunotherapy may involve multiple administrations of highly mutated mAbs, immunogenicity following two or three injections of plant-derived antibodies administered 2-3 weeks apart at 5-10 mg/kg, was also assessed in parallel with the pharmacokinetic studies. To measure the monkey anti-human immune responses against the injected mAbs, plasma or sera is tested at different times after each injection using an ELISA that employed a special anti-monkey secondary antibody (1B3) that does not react with human IgG. It should be noted that, like humans, macaques are outbred and genetics may play a role in the type of antibody response induced. The bnAbs presenting immunogenic epitopes are indicated in Example 8 and reference in FIGS. 9 and 10).

It was important to demonstrate that the antibodies produced against the epitopes on plant-derived bnAbs are similar to those epitopes produced in other expression systems and not specific for contaminants in the plant mAb preparations (e.g. host cell derived impurities), (Example 9 and referenced in FIG. 10).

Confirmation of the Anti-Idiotypic Specificity in the Sera of Macaques Injected with Each bnAb.

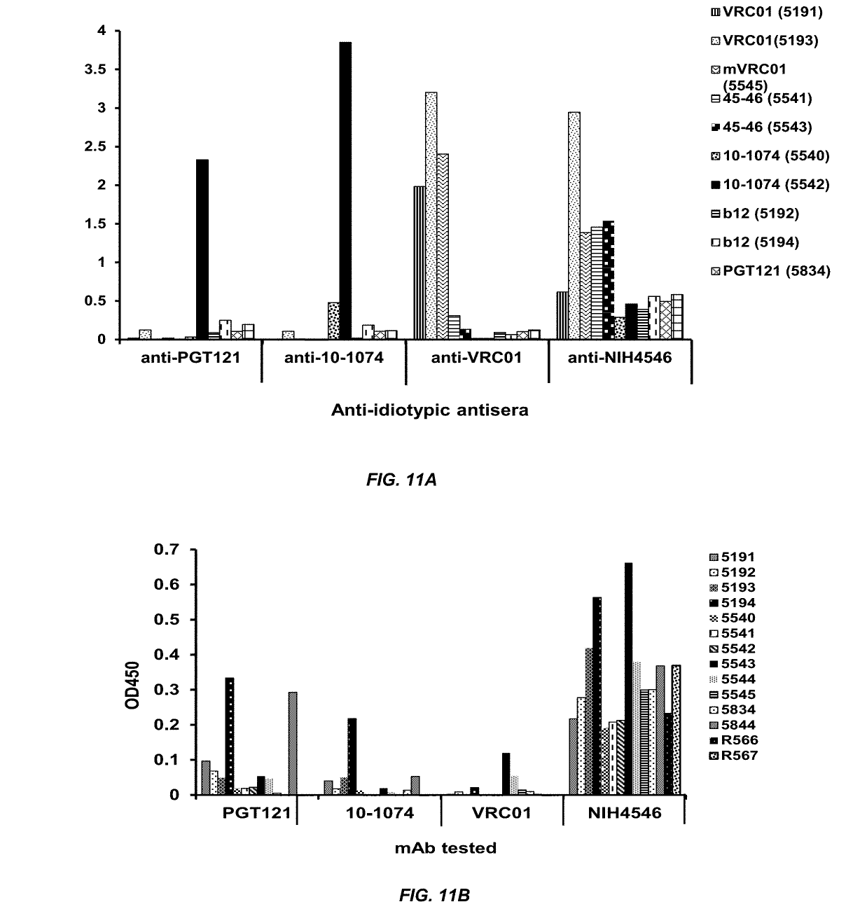

It was also important to confirm that anti-idiotypic antibodies produced in macaques against the injected bnAbs are specific for their cognate idiotypes and closely related idiotypes and do not react nonspecifically with any antibody. To assess this, sera from macaques injected with a particular bnAb were tested against each of the different antibodies both using binding assays and by a functional neutralization inhibition assay. (Example 10 and referenced in FIG. 11A.

During these studies, it became unexpectedly apparent that many macaques appeared to have pre-existing “anti-idiotypic” antibodies in their circulation prior to injection; presumably due to environmental stimulation. This was particularly true for one of the antibodies (mNIH45-46 G54W). To establish the different anti-antibodies in presumably naïve animals, macaques were bled and tested using an ELISA assay for the levels of anti-antibodies specific for many of the bnAbs under study (Example 10 referenced in FIG. 11B).

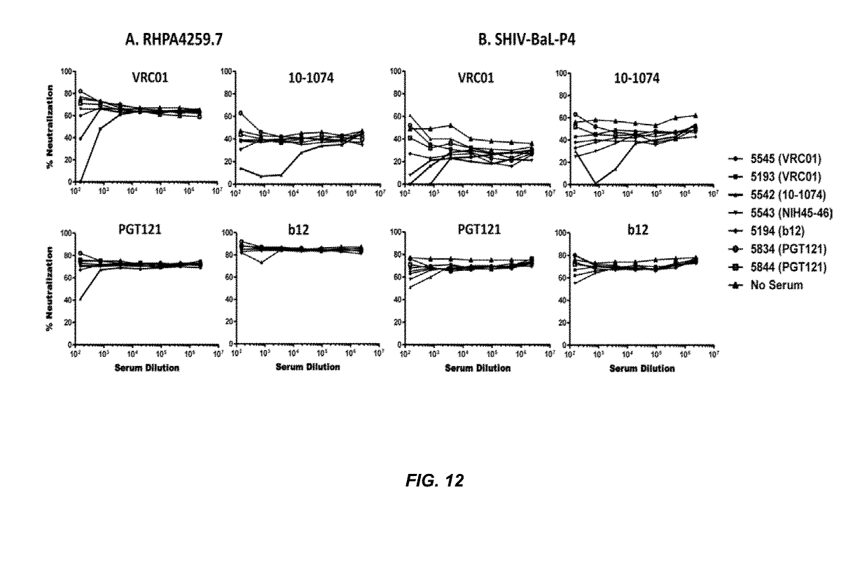

To corroborate the binding assays, monkey anti-idiotypic sera were also tested to show that they also could inhibit the activity of their cognate idiotypes following binding in a HIV neutralization assay i.e. whether the macaque anti-idiotypic antibodies could functionally inhibit their cognate idiotypes (Example 10 referenced in FIG. 12).

### Efficacy of Protection by Plant-Derived Delivered as a Cocktail or Singly.

In 2011, almost all of the 230,000 deaths from AIDS of the ˜330,000 infected children in Sub Saharan Africa, became infected as a result of mother-to-child-transmission (MTCT) (AVERT, 2014). While antiretroviral therapy (ARV) consisting of a cocktail of HIV drugs has greatly reduced the risk of MTCT, two out of three women in low and middle-income countries do not know their status and the proportion of women, in low and middle-income countries, accessing effective treatment for MTCT, stands at just over half. Moreover, even in mothers receiving ARV treatment, infection breakthrough occurs in 2-5% at the time babies are 6 months old and pregnant women who have received ARV treatment for <4 months are not protected from transmitting their virus to their babies. Thus, affordable passive immunotherapy will make an important contribution to the armamentarium of available treatments. In addition because of the unknown status of women coming into the hospital for delivery and a high incidence of HIV infection in women, it is advisable to treat all women and their babies at birth.

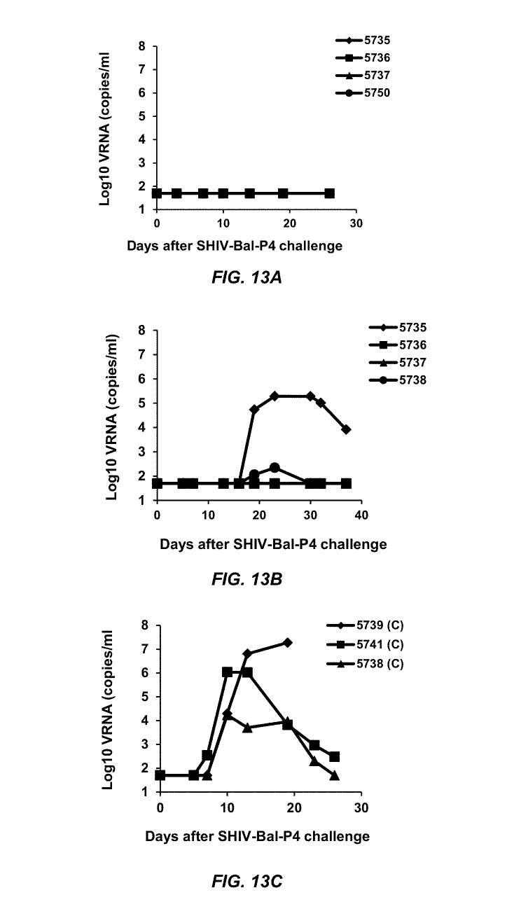

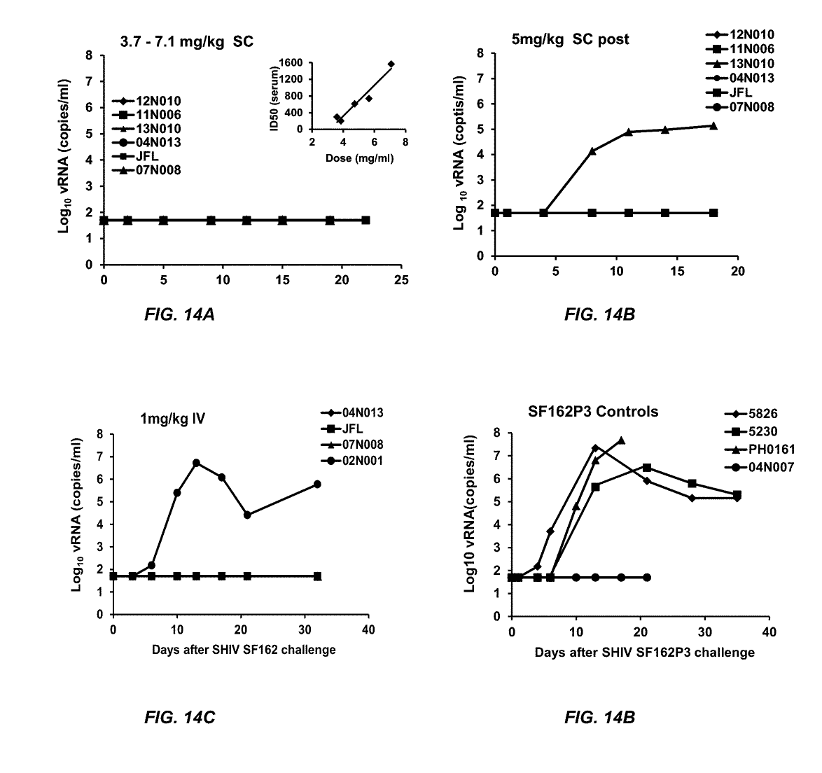

Based on levels of expression and their IC₅₀s against chimeric SHIV isolates, plant-derived bnAbs were delivered by different routes of administration both pre- and post-challenge with chimeric simian-HIV (SHIV) isolates to assess their ability to mucosally prevent infection of macaques by two different SHIV isolates delivered intrarectally and intravaginally (Example 11, FIGS. 13 and 14). Both unique cocktails and single antibody treatments have been investigated delivered both by IV and S C administration. SHIV is used because HIV does not replicate efficiently in monkeys.

### DISCUSSION

The production of >15 broadly neutralizing plant-derived HIV mAbs with IC50s equivalent to their mammalian counterparts for the first time in plants, demonstrates the versatility and speed of the transient plant expression system. While mAbs were initially chosen for further study based on high expression and potency, in certain cases, the properties of these mAbs determined in part by VH and VL mutations, long CDR H3, and/or plant-specific post-translational modification may significantly affect purification, solubility, plasma stability, and immunogenicity and impact their use as commercial therapeutics. Unique treatment cocktails of plant-derived bnAbs are now possible.

The importance of an L-chain glycan at N92 in determining clearance rates from the blood was seen during PK studies with VRC01. In this case, injection of either ˜5 mg/kg and 10 mg/kg of the WT exhibited very low Cmax at 30 mins and total clearance by 4 hr (insert in FIG. 6) compared with a typical dose dependent Cmax and greatly increased retention time when the L-chain glycan was eliminated (mVRC01M92T). Unlike the unexposed glycan at N297 in the CH2 chain domain of IgG antibodies, which to date has been shown to exert either no effect on clearance rates or a reduction in circulatory retention in the case of Man5 appended glycans, these results suggest that exposed L chain glycans terminating in GlcNAc, which make up a majority of plant-derived proteins, lead to rapid receptor-mediated removal. Interestingly, in contrast to the mVRC01, mNIH45-46^{G54W} (M92T) was cleared rapidly despite the elimination of the L chain glycan.

Unlike antibodies elicited through vaccination, the breadth and potency of broadly neutralizing mAbs have been shown to correlate well with their level of somatic mutation and the frequency of insertions. The 2-3 year-maturation process whereby these antibodies develop from germline to affinity matured antibody, and the consequences of eliciting these bnAbs, are only now being elucidated. In early studies, the presence of long hydrophobic CDR3 regions (22-24 aa), the direct binding of the prototypic bnAbs 2F5 and 4E10 to lipid autoantigens, and the association between acquisition of bnAb activity and auto-reactivity, suggested that one reason highly potent bnAbs are not more commonly seen is related to B cell tolerance mechanisms.

In the present studies, the lack of polyreactivity of these bnAbs (data not shown) and the anti-idiotypic antibody response induced in macaques following the second injection of the highly mutated VRC01 (differing from GL by 32% VH; 17% VL), 10-1074 (29% VH) and NIH 45-46 (40% VH; 26% VL) but not following two injections of b12 (˜14%) and PGT121 (34% VH; 28% VL) (FIG. 9) raises the intriguing possibility that the HIV-infected individuals who lack broadly neutralizing antibody in their circulation, may have actually generated such potent anti-HIV responses at some stage but may have subsequently induced an anti-idiotypic response leading to their elimination and overall reduced plasma neutralizing activity. Thus immunogenic eptiopes on HIV bnAbs, resulting from certain mutations, are very important but cannot be easily predicted.

Although the monkey anti-human responses could be directed to IgG Fc regions, it is unlikely since all of the plant-derived bn mAbs we have produced share the same CH and CL regions but do not equally induce anti-mAbs antibodies. In addition, it is unlikely that antibodies to plant-specific glycans e.g. beta1,2-xylose, alpha-1,3-fucose, play a role since all mAbs are produced in the same N. benthamiana hosts plants using the same glycosylation machinery. In addition, strong anti-VRC01 responses were observed when in vivo clearance was rapid (WT) or much slower (N92T mutation). Finally, the similar binding patterns on highly purified CHO or HEK-derived mAbs (FIGS. 9 and 10) precluded non-specific binding to plant non HIV proteins in the extract.

While a close association between the levels of mutation and immunogenicity was apparent with b12, mVRC01, 10-1074 and NIH45-46, the highly mutated PGT121 was not immunogenic after two or three injections two weeks apart. This lack of immunogenicity was interesting, since 10-1074 which belongs to the same family of antibodies as PGT121 and shares a common GL gene, induced a strong antibody response in 10-1074-injected macaques which cross-reacted with PGT121 by ELISA and to a lesser extent by neutralization inhibition. Based on the large number of common substitutions (50%) in the VL chains of these two molecules, it is possible that these residues represent the antigenic epitope/s on 10-10-74 and PGT121 responsible for the cross-reactivity in the ELISA assays, while the lack of immunogenicity of PGT121 may be a result of either specific mutations in the more diverse CDRVH, CDRL3 and framework regions or the absence of a T cell epitope in the PGT121. This once again highlights the importance of using a model of plant mAbs in macaques capable of assessing the generation of immunogenic epitopes and the induction of anti-idiotype antibodies in vivo.

In vivo testing with plant-derived NIH45-46^{G545W} has revealed that this bnAb has many features different than the other bnAbs tested. For example, most macaques appear to have varying levels of pre-existing antibodies that bind to mNIH45-46^{G545W} prior to any exogenous administration when tested on both plant- and CHO-derived forms.

The finding that a relatively high number of the bnAbs tested exhibited immunogenicity following injection in macaques, as well as the emergence of viral escape mutants in infected recipients preventing elimination even by an efficacious non-immunogenic bnAb, will necessitate rapid development of new potent antibody-based therapies. The recent production of 15 broadly neutralizing plant-derived HIV mAbs in the current study, highlights the unique advantages of the transient plant system in terms of speed and versatility, pathogen-free nature and low-tech requirements; particularly in the early developmental stages from “cloning to preclinical protection studies”.

Taken together, the current studies demonstrate that anti-idiotypic antibodies induced in monkeys are capable of strongly inhibiting neutralization of their cognate idiotypes and to a lesser extent their close family members. These findings in macaques, suggest that specific mutations in these bnAbs contribute to their immunogenicity and call attention to the prospect that mutated bnAbs will be immunogenic in humans, thereby reducing their value for prophylaxis and therapy of HIV-1 involving multiple administrations.

Immunogenicity of biological drugs such as HIV bnAbs is not only one of the parameters to be monitored following biological drug treatment, it can also be useful in the initial choice of the drug to be used in a specific subject as well as in the switch from one particular therapeutic to another. In this way, the present findings have indicated that for the initial treatment of e.g. pregnant mothers, any bnAb or cocktail of bnAbs may be used prior to birth but currently, except for PGT121 amongst those described here, different bnAbs will have to be used for each subsequent administration to the mother and subsequently to the baby to avoid the induction of anti-idiotypic antibodies and to maintain protection whilst breast feeding. Similar protocols will have to be followed for passive immunotherapy to control acute or chronic infections to prevent sexual transmission by the use of vaginal microbicides utilizing bnAbs.

### Example 1. A Method of Transient High Level Production of HIV MAb in Plants

### Plant Expression Vectors

The codon usage of the human kappa (AAA58989.1) (or lambda (AFR33667) constant light (CL) domains and human constant IgG1 heavy (AAC82527.1) (CH) domains were adapted to tobacco by introducing codons preferentially used in highly expressed tobacco genes. The constant domains were flanked by type-IIs restriction enzymes to allow seamless joining of the variable domains. The codon usage of the variable antibody domains were also modified as described above and flanked by restriction sites generating non-palindromic compatible ends with the target vectors. The recombinant antibody genes were then generated by ligating the BfuAI digested variable domains with the BfuAI digested vectors containing the respective constant domains. Constructs with a C-terminal SEKDEL tag were generated by deleting a Bbsl fragment. The plant expression vectors were generated by cloning the EcoRI-XbaI fragments into the T-DNA vector pTRA-k and verified by sequencing. A pTRA-k plasmid containing the tomato bushy stunt virus p19 inhibitor of silencing (TBSVp19, AJ288917) was co-transfected. The p19 genes from the cucumber necrosis virus (CNV) (AJ288922) and lettuce necrotic stunt virus (LNSV) (AJ288915) have also been used.

### Agrobacterium, Transient Transfection and Plant Cultivation

Transformation, selection and cultivation of Agrobacterium tumefaciens strain GV3101 (pMK90RK) and cultivation of tobacco plants in the greenhouse or in growth chambers was essentially performed as described previously. Briefly, recombinant Agrobacteria were cultivated in YEP medium with appropriate antibiotics, resuspended in MS medium to OD600 nm=0.5-1 and infiltrated either by injection or vacuum using 6-8 week old plants or leaves of Nicotiana bentamiana For co-infiltration experiments, amounts of Agrobacteria carrying the light chain, heavy chain and p19 expression plasmids (in the ratio 1.0:0.6:0.55 respectively) were mixed immediately before use. After infiltration plants were incubated at 20° C. with a 16/8 h day-night cycle for 3 to 16 days and either directly harvested or stored at −20 C. For initial screening using ELISA and Western blotting, six leaf discs (11 mg) were collected from different positions in the transfected leaves, minced with 200 ul PBS and centrifuged.

It should be noted that small plants and leaves of N. tabacum can be infiltrated; with the larger N. tabacum leaves being maintained in moist containers under lights until harvested.

A C-terminal SEKDEL tag on the bnAbs was used for ER retention to generate high mannose glycoforms to assess differences in levels of accumulation of the recombinant antibodies.

### Results

After 10-14 days incubation of the infiltrated plants soluble antibody proteins were extracted in buffers and under conditions as described in Table 1. During the final processing step of the leaf extract, buffers also contained chitosan (0.02%, for 1 hr with stirring). The bnAbs were purified by 2- or 3-step procedures using protein-A and MEP-hyperCel mixed-mode sorbent chromatography plus a final filtration, producing 100-1,300 mg of purified bnAb/kg of leaf biomass depending on the bnAb FIG. 1. demonstrates the expression level of PGT121 in serial diluted D12 leaf extract (2,000-31 ug) by Western blotting against know concentrations of a highly purified PGT121 and indicates that 1.3-1.6 g/kg of this bnAb is produced using the N.b./p19 system.

N92T mutated forms of VRC01 and NIH45-46 bnAb, in which the VL glycans were removed, were also expressed at levels similar to the non-mutated form.

TABLE 1

| mAb | Extraction Buffer | | Elution Buffer |
|---|---|---|---|
| PGT121 | NaH2PO4 | 0.05 M | 100 mM glycine pH3.5 |
| VRC01-mu | NaCl | 0.25 M | Raise pH with pH4.75 Na Acetate/ |
| | | | acetic |
| | Na2S2O5 | 0.005 M | Acid |
| | EDTA | 0.005 M | 80 ul/1.5 ml fraction |
| | pH5.5 | | |
| b12 | MES | 0.05 M | 100 mM glycine pH3.5 |
| | NaCl | 0.25 M | Raise pH with pH4.75 Na Acetate/ |
| | | | acetic |
| | Na2S2O5 | 0.005 M | Acid |
| | EDTA | 0.005 M | 80 ul/1.5 ml fraction |
| | pH5.5 | | |
| 10-1074 | NaH2PO4 | 0.01 M | 100 mM glycine/100 mM fructose |
| | | | pH3.2 |
| | Na2HPO4 | 0.04 M | Raise pH with pH8.0 1.0 M TRIS |
| | Na2S2O5 | 0.005 M | 100 ul/1.5 ml fraction |
| | NaCl | 0.5 M | |
| | pH7.5 | | |
| NIH45-46 | NaH2PO4 | 0.01 M | 100 mM glycine/100 mM fructose |
| | | | pH3.2 |
| | Na2HPO4 | 0.04 M | Raise pH with pH8.0 1.0 M TRIS |
| | Na2S2O5 | 0.005 M | 100 ul/1.5 ml fraction |
| | NaCl | 0.25 M | |
| | pH7.5 | | |

### Example 2. High Neutralization Activity of Plant-Derived HIV bnAbs

Neutralizing antibody assays were performed in TZM-bl cells as previously described using purified plant-derived recombinant bnAbs. These antibodies were tested starting at 50 μg/ml with serial 3-fold dilutions. Plasma (both heat-inactivated and non-heat-inactivated) was tested starting at a 1:20 dilution. Diluted test samples were pre-incubated with pseudovirus (150,000 relative light unit equivalents) for 1 hr at 37° C. before addition of cells. Following 48 hr incubation, cells were lysed and luciferase (Luc) reporter gene activity determined using a microtiter plate luminometer and BriteLite Plus Reagent (Perkin Elmer). Neutralization titers are the sample dilution (for plasma) or antibody concentration (for purified mAb) at which relative luminescence units (RLU) were reduced by 50% compared to RLU in virus control wells after subtraction of background RLU in cell control wells.

### Results

Neutralization activity of KDEL-tagged (usually high mannose) and non-KDEL-tagged (usually complex) glycoforms of N.b/p19-derived mAb was measured in a TZM-bl assay as a reduction in Luc reporter gene expression after a single round of infection with a small panel of Env-pseudotyped viruses, including six Tier 2 HIV-1 isolates (FIG. 2A) and three simian/human HIV (SHIV) isolates (FIG. 2B). Several bnAbs produced in mammalian cells (VRC01(H) produced in HEK293 cells, PGT128 (C) produced in CHO cells, and CHO1-31, a pool of two mAbs, known to efficiently neutralize most HIV variants) were used as positive controls. Overall, with the exception of NIH45-46G54W, all plant-derived bnAb IC50s were similar to control mAbs; PGT121 exhibiting the highest potency with an IC50 of <0.01 against 8 out of 9 HIV and SHIV isolates. Surprisingly, the high mannose glycoforms, 10-1074-KDEL and NIH45-46G54W-KDEL had lower neutralization activity than the non-KDEL complex glycoforms. Potency against SHIV isolates SF162P3, 1157ipd3N4 and SHIV-Bal-P4 was assessed to determine the most appropriate challenge isolate for macaque passive protection studies. 10-1074 and PGT121 had the lowest IC50 against the clade C 1157ipd3N4. These data indicate that any bnAb made in the more rapid, adaptable and less expensive plant expression system, exhibit the same or better IC50 as those produced in other systems.

### Example 3. Plant-Produced b12 has Higher Neutralizing Activity (Lower IC50) than its Counterpart Derived from Mammalian Cells

To demonstrate the feasibility of producing functional glycoforms in plants and assess their neutralizing activity (IC50s), high mannose and secreted complex glycoforms of the well characterized HIV bnAbs were produced transiently in Nicotiana tabacum and N. benthamiana to assess their IC50s against various viral isolates. The general rule is that KDEL, being an ER-retention signal, will result in KDEL-tagged proteins containing almost all oligomannose-type (OMT) glycans which will remain in the endothelium reticulum (ER) and not enter the golgi where complex glycans are appended.

### Results

KDEL and non-KDEL glycoforms of HIV bnAbs produced in the Nb/p19 plant system generally exhibit IC50s against a panel of HIV isolates similar to the control mAbs produced in mammalian cells and with no consistent differences in activity between the high mannose or secreted glycoforms (Rosenberg), with exceptions being seen in FIG. 2. Analyses of the KDEL and non-KDEL b12 plant-derived glycoforms indicated that (i) the IC50s of complex b12 glycoforms (solid black) were lower (increased potency) than the CHO controls ((striped) against 8 HIV isolates tested and (ii) the high mannose KDEL-tagged b12 glycoproteins (crossed) were 4-10 less potent than the complex b12 glycoforms (FIGS. 3A and 3B)

### Example 4. Binding of Plant-Derived b12, VRC01 and 2G12 to CD64/FcgRI by Surface Plasmon Resonance Analysis (SPR, Biocore)

Since no primary sequence differences were observed in the plant- and CHO-derived b12, the surprising small but significant decreases in the IC50s of plant-derived b12 likely reflects atypical plant glycosylation specifically in the case of b12. To initially assess this, binding studies using plant-derived b12 and VRC01 to the soluble extracellular domain of FcgRI/CD64 were performed, since Fc-Fcy1R interactions are commonly affected by the N-glycosylation of Asn297 in the CH2 domain of IgG H chains and because these assays have previously proven useful in terms of understanding the synthesis, trafficking, glycosylation and function of recombinant bnAbs.

### Results

FIG. 4 shows binding studies using surface plasmon resonance (SPR), of different samples of b12 glycoforms, harvested and purified at different times post infiltration, with rsFcyR1/CD64. Compared to the CHO-derived 2G12, H10 and b12 mAbs, the plant-derived b12 antibody preparations show significantly lower CD64 binding capacity and faster dissociation kinetics; the non-KDEL samples from the longer D11 and D14 incubation times exhibiting the weakest binding. These results indicate that, since b12 has no potential glycosylation sites in the VH or VL regions, the glycans appended to the N297 are undergoing changes on both the KDEL- and non-KDEL-tagged molecules during the D4 to D14 incubation period in the plants and which markedly influence Fc/FcRg1 interactions. Very similar abnormal rsFcyR1/CD64 binding was observed with plant-derived VRC01 glycoforms harvested at similar times post infiltration.

### Example 5. Glycosylation Profiles of Plant-Derived HIV b12 and VRC01 Monoclonal Antibodies

Surprisingly, binding kinetics of plant-derived b12 and VRC01 to both HIV gp120 envelope (variable region) and FcR (constant region) differed from other HIV bnAbs and prompted a more detailed comparison of the synthesis and N-linked glycosylation of several non-KDEL and KDEL bnAb glycoforms. As noted, KDEL is used as an ER-retention signal, resulting in KDEL-tagged proteins containing almost all oligomannose-type (OMT) glycans which remain in the ER. In addition, binding of IgG1 antibodies to soluble FcyRI is affected by N-glycans attached to Asn297. An analysis of the glycosylation profiles of non-KDEL and KDEL-tagged b12 and VRC01 glycoforms were therefore performed by mass spectrometric analysis. Plant produced 2G12-KDEL and PG9-KDEL were used as controls.

### Results

FIG. 5 shows that while the 2G12-KDEL and PG9-KDEL both have predominantly OGM (Man 7-9) glycans as expected, b12-KDEL and VRC01-KDEL glycovariants contain predominantly complex glycans with very small percentages of OGM glycoforms. In addition, in contrast to the GlcNAc₂Fuc1Xyl₁Man₃GlcNAc₂ usually predominant in plants, a high percentage of the complex glycans appended to the b12 were typically vacuolar with one particular HexNAc₁GlcNAc₁Fuc₁Man₂GlcNAc₂ structure which is presumably vacuolar due to its very trimmed form, which has not been previously described to our knowledge. In one case, this unusual glycan represented 23% of the total b12 glycans.

The glycans on VRC01 and VRC01-KDEL, while different from b12, were also atypical of the glycosylation profiles observed on plant-derived mAbs. Thus, both were predominantly GlcNAc₂ Fuc1Xyl₁Man₃GlcNAc₂ unlike b12. This results were quite unexpected and indicate that these two plant derived antibodies differ considerably from other plant-derived antibodies in at least two ways; in contrast to the high mannose glycans usually appended to proteins carrying the KDEL tag for ER retention, the KDEL-linked b12 and KDEL-VRC01 proteins contained predominantly complex glycans indicating that they had passed through the Golgi compartment despite having a C-terminal KDEL tag. In addition, the non-KDEL and KDEL-tagged VRC01 and b12 antibodies also escaped from the cis-Golgi and harbored plant-specific vacuolar N-glycans.

### Example 6. Pharmacokinetics of Plant-Derived HIV bnAbs

To assess the clearance rates of several the plant-derived bnAbs and their potential for in vivo therapeutic use, plant-derived VRC01, NIH45-46^{G54W}, b12, 10-1074, PGT121 and 10E8 were injected I.V. at 5, 7.5 or 10 mg/kg into each of two macaques to assess in vivo stability. Circulating levels of each mAb were assessed in two ways either by ELISA or by neutralization activity.

Firstly, to monitor the rates of clearance of the circulating bnAbs by ELISA, 96-well Immuno Module plates (Nunc) were coated with purified plant-derived (high mannose) HIV 89.6P gp140deltaCFI-KDEL (1 μg/ml) and incubated for 2 hr at RT with serial dilutions of leaf extracts or purified plant- or mammalian cell-derived mAbs. In some cases e.g. detection of PGT121 levels, wells were coated with anti-human kappa LC (50 mL of 1 μg/mL) (K3502, Sigma) or with either CHO-derived monomeric HIV BaL-gp120 (NIH HIV Reagent Program) or m.CONgp140 env (a kind gift of Dr Bart Haynes, Duke Univ., NC) which contain mammalian complex glycans required for binding. Control HEK-293 VRC01 was kindly provided by the VRC, NIH and the CHO-derived PGT121 by IAVI, NY. Wells were blocked with 5% (w/v) milk in PBST, washed 3-5 times with PBST, incubated with a 1/8,000 dilution of peroxidase-labeled goat anti-human IgG (Fc) (A0170, Sigma), and developed with tetramethylbenzidine (TMB) liquid substrate system (T0440, Sigma). Reactions were stopped with 0.5 N H2SO4, and endpoints were determined at 450 nm using the SPECTRA max PLUS plate reader (Molecular Devices).

Secondly, levels of neutralizing bnAbs in the circulation were assessed using the TZM-bl assay with plasma samples collected from macaques at different time following injection of the bnAbs. Purified recombinant antibodies were tested starting at 50 μg/ml with serial 3-fold dilutions. Plasma (both heat-inactivated and non-heat-inactivated) was tested starting at a 1:20 dilution. Diluted test samples were pre-incubated with pseudovirus (˜150,000 relative light unit equivalents) for 1 hr at 37° C. before addition of cells. Following 48 hr incubation, cells were lysed and luciferase (Luc) reporter gene activity was determined using a microtiter plate luminometer and BriteLite Plus Reagent (Perkin Elmer). Neutralization titers are the sample dilution (for plasma) or antibody concentration (for purified mAb) at which relative luminescence units (RLU) were reduced by 50% compared to RLU in virus control wells after subtraction of background RLU in cell control wells.

### Results

Despite known animal variability, most plant-derived bnAbs exhibited good Cmax levels of ˜120-150 ug/ml in the circulation ˜1 hr following a 5 mg/kg i.v. injection; consistent with plasma representing ˜4% of the body weight. FIGS. 6A and 6B indicates that the pharmacokinetics of clearance of b12 (at ˜5 mg/kg and 7.5 mg/kg), PGT121, 10-1074 and 10E8 (each 5 mg/kg) was in the observed Cmax range and dose-dependent. However, in the case of PGT121, while some monkeys injected IV with 5 mg/kg exhibited pharmacokinetics similar to those in FIG. 6A, other monkeys exhibited rapid clearance when monitored using either ELISA (ug/ml) and ID50 in TMZ-bl neutralization assays (FIG. 6B) possibly indicating an important role for primate genetics. Interestingly, while 10-1074 showed typical clearance rates, NIH45-46^{G54W} was rapidly eliminated from the circulation (FIG. 6C)

### Example 7. Elimination of V Region Glycans to Increase Plasma Retention (FIG. 7)

An unexpected finding in the pharmacokinetic studies was that, compared to other plant-derived bnAbs, VRC01 and the closely related NIH45-46^{G54W} produced in plant cells were more rapidly cleared from the circulation (FIG. 6B and see insert, FIG. 7A).

Although all HIV bnAbs contain a glycan at N297 in the CH2 domain, VRC01 and the closely related NIH45-46^{G54W} also contained a VH region glycan which was eliminated by site specific mutagenesis using a polymerase chain reaction (PCR) to assess the effects on plasma stability. Oligonucleotides NX156 (5′ AGCAGTAC CAA AGC ACG TAC CGG GTG 3′) and NX157 (5′ TACGTGCTTTG GTA CTG CTC CTC 3′) were synthesized (DNAgency, Malvern, Pa.) to anneal to the coding (NX156) and noncoding (NX157) strands of the human heavy chain gene over the region containing the N-glycosylation site (Asn-Ser-Thr). Both oligonucleotides contained a two-base mismatch designed to mutate a codon from AAC (asparagine) to CAA (glutamine), In the first round of PCR, NX156 was paired with a downstream primer NX113 (5′ GCTGACGGAT CCTCATTTAC CCGGAGACAG GGAG 3′) and NX157 was paired with an upstream primer NX110 (5′ CCGTCTATTA CTGTTCTAGA GAGGTC 3′) in separate reactions using plasmid PNRX451 as a template and DNA polymerase according to the manufacturer's specifications (Perkin Elmer, Branchburg, N.J.). The resulting PCR products were 476 base pairs for the NX110/NX157 primers and 634 base pairs; for the NX156/NX113 primer pair; and comprised portions of the heavy chain extending upstream and downstream from the mutation, respectively. These PCR products were purified from agarose gels via Geneclean (Biolol, Vista, Calif.) and combined into a contiguous fragment in a second PCR using primers NX110 and NX113. The resulting PCR product was 1190 base pairs and contained the desired mutation. The product was digested with restriction enzymes Sstll and BamHI to generate a 471 base pair fragment which was cloned into the expression vector pNRX451, replacing the N-glycosylation site containing wildtype gene fragment.

### Results

The elimination of the N-linked glycan at N92 on the VL chain of VRC01 (now termed mVRC01) greatly increased plasma stability (FIG. 7A). Thus, following injection, mVRC01 now exhibited dose-dependent Cmax of ˜260 ug/ml and 160 mg/ml in the circulation 1 hr following i.v. injections of 10 mg/kg and a 5 mg/kg i.v. respectively. In addition to the ELISA assays, plasma samples from macaques injected with 10 mg/kg of mVRC01 (ug/ml) were also tested for serum neutralizing activity (ID50) using a pseudovirus/TZM-bl assay (FIG. 7B). These results indicated a close correlation between the serum mAb concentrations of the plant derived bnAbs determined by both binding and neutralization activity.

Interestingly, mutation of NIH45-46^{G54W} (mNIH45-46^{G54W}) a close VRC01 family member did not increase plasma retention. Thus, macaques injected with 5 mg/kg m454-46^{G54W} exhibited a normal Cmax but rapidly cleared the mAb from the blood by 4 hr despite the N92T mutation (referenced in FIG. 6B).

Similarly, modification of the O linked glycan in the H chain of PGT121 may also increase plasma stability.

### Example 8. Plasma Stability of Plant-Derived bnAb Administered S.C. and I.M.

I.V. routes of delivery, especially in the case where multiple administrations are required and in locations are not easily accessible eg people in rural area of Africa, are not practical and I.M. injections can have variable results. This is especially relevant for babies where neither I.V nor I.M. delivery of treatments are appropriate.

In order to assess the Tmax and thus the optimal time to challenge the treated macaques following different routes of delivery, two African green monkeys were each injected subcutaneously (S.C.) and intramuscularly (I.M.) with 5 mg/kg and the clearance profiles determined. To examine clearance profiles, blood samples were monitored from 4 hr to 14 days and assessed for neutralizing activity (ID50) using a pseudovirus/TZM-bl assay (FIG. 8), by ELISA to determine peak concentration (ug/kg) and to determine the pharmacokinetic parameters time of peak levels (Tmax), area-under the curve (AUC), mean retention time (MRT) and terminal half-life (T½)(using a Windows-based PK Solutions 2.0 program, a non-compartmentalized analysis of pharmacokinetic data). These values were compared to the clearance following an IV injection with 5 mg/kg administration.

### Results

Referring now to FIG. 8, it compares the serum ID50 clearance profiles for each monkey injected SC and IM using a pseudovirus-based TZM-bl assay against SHIV SF162P3 while Table. 2 displays their matching pharmacokinetic parameters (terminal half-life (T½), peak concentration (Cmax) m Area under the curve (AUC) and mean retention time (MRT) determined by ELISA, including an historic 5 mg/kg PGT121 IV control (from Example 5 above). Results indicate that each of the monkeys receiving SC injections (#8338, #8291) exhibited similar PK parameters with Cmax of 59 μg/ml and 79 μg/ml at Tmax of ˜24-30 hr. By contrast, clearance profiles and Tmax in monkeys injected IM differed widely. Thus, monkey #8288 exhibited a T max of 4 hr and a Cmax of 110 ug/kg similar to the Cmax following IV delivery (115 μg/ml) while the corresponding values for #8390 were 16 μg/ml at a Tmax ˜18-24 hr. Analysis of pharmacokinetics by ELISA in Table 2 revealed very similar clearance profiles with Cmax values of 59 and 79 μg/kg for both macaques (#8338 and 8291). Based on minimum predicted protective neutralizing titers, these data also suggest that SC injection of PGT121 (5 mg/kg) will be protective within a range of <4 hr to >14 days since the T½ is considerably longer after SC and IM than IV administration.

TABLE 2
Pharmacokinetic parameters in macaques following SC, IM and IV
injections of PGT121 bnAb.

| Parameter | SC 8338 | SC 8291 | IM 8288 | IM 8390 | I.V. |
|---|---|---|---|---|---|
| E Half-life (hr) | 143 | 157 | 211 | 156 | 88 |
| Cmax (obs) (ug/ml) | 59 | 79 | 110 | 16 | 115 |
| AUC¥ (ug-hr/ml) | 7,612 | 10,680 | 6,796 | 2,540 | 7,412 |
| MRT (hr) | 168 | 200 | 208 | 208 | 92 |

Two monkey each injected SC, IM and IV. The data from the latter IV macaques are averaged and represent those shown in clearance profiles in FIG. 6A.

### Example 9. Assessment of Immune Responses in Macaques Against the Injected Plant-Derived with bnAbs

Since passive immunotherapy may involve multiple administrations of highly mutated mAbs, immunogenicity was also assessed in parallel with the pharmacokinetic studies following two or three injections of plant-derived VRC01, mVRC01, 10-1074, NIH45-46G54W, b12 and PGT121 bnAbs (5-10 mg/kg) administered 2-3 weeks apart. To measure the monkey anti-human antibody responses against the injected bnAbs, plasma was tested at different times after each injection using an ELISA that employed an anti-monkey secondary antibody (1B3) that does not cross react with human IgG.

The pharmacokinetics and immunogenicity of the administered bnAbs were assessed using an ELISA to measure specific binding and a neutralization inhibition assay.

To monitor the presence of a macaque antibody responses against the injected human HIV bnAbs, ELISA plates were coated at RT with the target antibodies at 1.2 μg/ml for 2-4 hr. Following incubation, wells were blocked, washed and incubated for 2 hr with monkey plasma or serum samples at 1/500 and 1/2500 dilutions followed by a third 2 hr incubation with 1/4,000 of a special mouse anti-macaque IgG (1B3-HRP).

### Results

Three types of responses occurred: 1. No response, which indicated that the particular bnAb is not highly immunogenic. 2. Primary responses at 3-14 days after the first injection reflecting possible polyreactivity or environmental stimulation and 3) Clear secondary anti-human IgG responses induced 7-21 days after the second injection. In the first immunogenicity study, in which two macaques each received either 4.5 and 7.5 mg/kg of b12 (#5192, #5194) or 4.5 or 10 mg/kg of VRC01 (#5191, 5193), all three types of responses were evident. FIG. 9A shows the binding of plasma from each of the four animals to both b12- and VRC01-coated wells and demonstrates that while b12 exhibited no/low anti-b12 responses, both macaques receiving VRC01 made a substantial anti-VRC01 antibody response at 14-21 days following the second administration. These anti-VRC01 antibodies did not cross react with b12 although plasma from macaque #5193, which appeared to be previously stimulated, did exhibit some cross-reactivity with b12. (FIG. 9A)

In the second immunogenicity study, the potent highly mutated bnAbs 10-1074 and mNIH45-46G54W were also tested with VRC01 for immunogenicity in macaques following two i.v. injections of 5 mg/kg 2-3 weeks apart. FIG. 9B shows that except for macaque #5544, which was injected with mVRC01, each monkey exhibited moderate to high anti-human bnAb responses 7-14 days after the second injection (FIG. 9B). Again, none of the plasma from these 6 macaques reacted with b12. Interestingly, the potent highly mutated PGT121, elicited no anti-antibody response against PGT121 following three injections of 5 mg/kg into two macaques (study #1) and two injections into two macaques (study #2) despite belonging to the same family as 10-1074 (data not shown). In these studies, monkey plasma that cross-reacts with 10-1074 was used as a positive control.

### Example 10. Immunogenic Epitopes on Plant-Derived bnAbs Induce an Idiotypic Antibody that Binds Equally to Plant- and CHO-Derived Antibodies

In order to demonstrate that the anti-human antibody response was against epitopes on the variable region of the mAbs and not specific for contaminants in the plant bnAb preparations (e.g. host cell derived impurities), plasma from all 6 of the VRC01-injected macaques were also tested by ELISA using highly purified HEK293-derived VRC01. Plasma collected at different times from VRC01-injected macaques exhibited identical binding to wells coated with either highly purified HEK293- or plant-derived VRC01 demonstrating that the observed responses were indeed specifically directed at the bnAbs.

### Results

FIG. 10 shows that sera collected at different times from VRC01-injected macaques exhibited identical binding to wells coated with either HEK-293 or plant-derived forms making it unlikely that contaminants in the latter were responsible for the binding and strongly suggesting the induction of anti-idiotypic antibodies.

### Example 11. Confirmation that Anti-Idiotypes Against the Plant-Derived bnAbs are Specific for their Cognate Idiotypes and Closely Related Family Members

To confirm the anti-idiotypic specificity of the antibodies induced in macaques following 2 injections of VRC01, mVRC01, 10-1074 and mNIH45-46^{G54W}, the positive sera from the macaques collected at 14-21 days after the second injection of each bnAb, were tested firstly using an ELISA against highly purified mammalian cell bnAbs e.g. HEK-293-derived VRC01 and CHO-derived 10-1074, NIH45-46^{G54W} and PGT121.

Secondly, the anti-idiotypic reactivity was tested by inhibition of neutralization In which dilutions of each of 7 sera from monkeys injected twice with VRC01, 10-1074, b12 and PGT121 were assessed for their ability to inhibit the neutralization activity of each of the four bnAbs when tested against HIV RHPA4259.7 and SHIV-Bal-P4 isolates in a pseudovirus/TZM-bl assay. A concentration of mAb that inhibited the target virus at 50-80% was pre-incubated with or without serial dilutions of monkey plasma samples for 1 hr at 37° C. prior to adding virus. After an additional 1 hour incubation of mAb/serum/virus, cells were added and the assay was continued according to the standard protocol. The ‘No Serum’ control indicates the level of mAb inhibition of virus. Deviations from this line indicates interference from the plasma sample with the neutral-ization of the bnAbs.

In addition, 14 naïve macaques were also screened against the same HEK-293-derived VRC01 and CHO-derived 10-1074, NIH45-46^{G54W} and PGT1 to determine whether animals had pre-existing levels of anti-antibodies due to environmental stimulation.

### Results

The results in FIG. 11A indicate the plasma from each macaque binds strongly only to the specific mAb it received with any cross reactivity observed being predictable based on the clonal families to which they belong. None showed significant binding to b12 or 10E8 (not shown). Thus, sera from macaques administered VRC01 or mVRC01 bound only to VRC01 and the clonally related NIH45-56^{G54W}. However antibodies induced by two injections of NIH45-46G54W bound strongly to VRC01, moderately to itself and weakly albeit significantly, to all bnAbs tested; suggesting that these anti-idiotypic antibodies were directed to either the TARDY insertion in the CDR3 region and/or the 6-amino acid changes in the CDR1 and CDR2. In the case of monkeys injected with 10-1074, plasma antibodies were once again shown to be specific for both 10-1074 as well as the related family member PGT121 with no/low reactivity to the other mAbs tested. Importantly, these anti-idiotypic antibodies showed similar binding patterns both either mammalian- and plant-derived mAbs.

Surprising, similar to the cross-reactivity previously observed (FIG. 9), most monkeys tested had low to moderate levels of antibodies specific for NIH45-46G54W and (ii) some macaques e.g. #5191, 5194 and 5844 bound at varying levels to several of the mAbs tested (FIG. 11B). This once again reflects the different genetic backgrounds in the macaques and the unexpected levels of anti-antibodies induced in “normal” monkeys.

FIG. 12 demonstrates that the inhibition by the anti-idiotypic antibodies was highly specific, dose dependent and was observed against both isolates. Thus, as observed in the binding assays, (i) anti-VRC01 blocked neutralization of both isolates by VRC01, although only significantly cross-blocked NIH45-46 neutralization of the SHIV-Bal-P4. (ii) anti-10-1074 inhibited strongly 10-1074 and to a lesser extent PGT121 and (iii) sera from PGT121- and b12-injected macaques did not inhibit their cognate bnAbs.

### Example 12. Efficacy of Protection of Plant-Derived bnAbs Against Mucosal SHIV Challenge

To assess protection in rhesus macaques, animals must be challenged with a SHIV isolate (a chimeric simian-HIV (SHIV). The IC50s of plant-derived bnAbs against SHIV isolates (FIG. 2) are therefore important in determining the SHIV used for challenge. While 10-1074 and PGT121 efficiently neutralized all SHIVs tested (SHIV-Bal-P4, SHIV-162P3, SHIV-162P4 and SHIV1157ipd3N4), VRC01, b12 and 10E8 were effective against SHIV-Bal-P4 but did not efficiently neutralize the SHIV 162P3 isolate. Based on levels of expression of 200-1,300 mg/kg of leaf biomass (FIG. 1), their potent and broad neutralizing activity against HIV Tier 2 HIV isolates and chimeric SHIV isolates (low IC50s) (FIG. 2) and their pharmacokinetics (FIG. 9), plant-derived bnAbs were delivered by different routes of administration both pre- and post-challenge with different SHIV isolates to assess their ability to mucosally prevent infection of macaques by two different SHIV isolates delivered intrarectally (IR) and intravaginally. Both unique cocktails and single antibody treatments have been investigated delivered both by IV and S.C. administration.

In the first study, a cocktail of plant-derived VRC01, 10-1074, b12 and 10E8 were tested for for their ability to protect prophylactically against intrarectal SHIV challenge (FIG. 2). Thus initially bnAb cocktail was injected IV at 5 mg/kg each (total 20 mg/kg), 6 hr prior to challenge with SHIV-Bal-P4; the high ID50 of the cocktail in the 4 pretreated macaques (#5735, 5736, 5737, 5750) being 7,755, 8,886, 8950 and 12,970 respectively. (FIG. 13A). After several weeks, a cocktail of VRC01, b12 and 10E8 (no 10-1074) at 5 mg/kg (total 15 mg/kg) was again administered IV 6 hr post challenge with SHIV-Bal-P4 and completely protected 2/4 with 1/4 being only transiently infected (FIG. 132B). SHIV Bal-P4 was kindly provided by Dr Sampa Santra, Center for Virology and Vaccine Research, Harvard Medical School for these studies.

In the second study, PGT1212 was used singly because of its potency, its lack of immunogenicity and its very high expression (1.3 gm/kg of leaf biomass). Six macaques received SC 5 mg/kg of PGT121 24 hr prior to 1,700 TCID of SHIV SF162P3 intra-vaginally. In order to mimic aspects of MTCT wherein infants born to HIV+ mothers may be injected immediately after birth, three months later these same six protected animals were administered 5 mg/kg of PGT121 SC immediately (30-60 mins) after intravaginal challenge with 1700 TCID SF162P3. Four control macaques were infected in the absence of bnAb.

### Results

In the first protection study using a cocktail of four plant-derived bnAbs mVRC01, b12, 10-1074 and 10E8 at 5 mg/kg (total 20 mg/kg), all 4/4 of the monkeys were protected against IR SHIV challenge when given 6 hrs prior to virus (FIG. 13A). Several weeks later the same four macaques were administered IV a cocktail of mVRC01, b12 and 10E8 at 5 mg/kg (total 15 mg/kg) at 6 hr post challenge with SHIV-Bal-P4; resulting in complete protection in 2/4 and only transient infection in 1/4 macaques (FIG. 13B). Three of 15 positive control macaques infected IR with the SHIV BalP4 are shown in FIG. 13C.

In the second protection study, the efficacy of SC-injected PGT121 to protect against SHIV SF162P3 challenge was assessed in an initial dose-finding study. Thus, doses of 3.5-7.1 mg/kg, administered at 24 hr prior to intra-vaginal challenge with SF162P3 (1700 TCID), resulted in sterilizing immunity in all 6/6 macaques (FIG. 14A). This is consistent with the serum PGT121 neutralizing levels at all doses against SHIV-SF162P3, measured just prior to challenge (insert in FIG. 14A) with the lowest dose of 3.5 mg/kg resulting in an ID50 of 1:295 (12N010) and reaching an ID50 of 1:1,565 at the 7.1 mg/kg dose. In the macaques that were injected with 5 mg/kg of PGT121 immediately after intravaginal challenge with 1700 TCID, good protection was also observed with only 1 of the 6 macaques becoming infected (FIG. 14B).

In order to show efficacy of protection against intravaginal challenge when administered IV, plant-derived PGT121 at was administered IV at 1 and 5 mg/kg, 6 hours prior to SHIV-162P3 (1700 TCID). FIG. 14C indicates that 3/4 macaques were protected even at the 1 mg/kg dose. As observed previously in several reports, one of the four untreated SHIVSF162P3-infected controls was not infected even at a high TCID (FIG. 14C).

These results indicate that the rapidly produced plant-derived bnAbs will be very good candidates for use as immunoprophylactic or immunotherapeutic treatments either singly or as a member of a cocktail.

### Definitions

Antibody encompasses fragments, fusions and other derivatives of antibodies.

Plant-derived antibody is an antibody that has been produced by heterologously expressing recombinant genes encoding at least one variable light or heavy chain domain in one or more plant cells.

While it is apparent that the invention herein disclosed is well calculated to fulfill the objects above stated, it will be appreciated that numerous modifications and embodiments may be devised by those skilled in the art. It is intended that the appended claims cover all such modifications and embodiments as fall within the true spirit and scope of the present invention.

1. A method of producing a set of one or more monoclonal antibodies in plant cells comprising the steps of:
(i) co-transforming a plant cell, plant tissue, plant organ or whole plant with gene expression constructs encoding an antibody variable heavy and light chain domain to form an infiltrated plant cell, plant tissue, plant organ or whole plant;
(ii) incubating the infiltrated plant cell, plant tissue, plant organ or whole plant for a period of 3 to 21 days;
(iii) homogenizing the infiltrated plant cell, plant tissue, plant organ or whole plant in the presence of an aqueous buffer;
(iv) isolating an aqueous extract from the homogenized infiltrated plant cell, plant tissue, plant organ or whole plant;
(v) supplying an additive to the aqueous extract
(vi) recovering one or more monoclonal antibodies at high yield.

2. The method of claim 1, wherein the monoclonal antibody has an accumulation level of at least 100 mg per kg of plant biomass, preferably more than 500 mg/kg, more preferably more than 750 mg/kg and most preferably more than 1000 mg/kg.

3. The method of claim 1, wherein the infiltrated plant cell, plant tissue, plant organ or whole plant is first incubated for a period of 1-10 days in N. tabacum, or for 2-21 days in N. benthamiana, at temperatures between 18-25° C.

4. The method of claim 1, wherein the supplied additive improves the downstream processing of the aqueous extract containing the antibody by improving the clarification, filtration, chromatography, product stability or product quality.

5. The method of claim 4, wherein the additive is a chitosan.

6. The method of claim 5, wherein the chitosan is added at a concentration range selected the group consisting of: 0.01-20 g/L, 0.01-5 g/L, 0.01-2 g/L and 0.01-0.5 g/L.

7. The method of claim 1 wherein at least one monoclonal antibody comprises an intrinsic targeting signal capable of overriding a C-terminal ER retrieval signal.

8. A set of one or more monoclonal antibodies produced in plant cells, having an improved property which is a result of an altered N- or O-glycosylation.

9. A monoclonal antibody of claim 8, wherein at least one human monoclonal antibody has been mutated to eliminate an N- or O-linked glycan within the variable heavy or light chain domain (and wherein the mutated form has an increased plasma stability).

10. A method of assessing the immunogenicity of antibody heavy or light chain variable domain epitopes (idiotypes) on a set of one or more monoclonal antibodies, comprising the steps of
a) injecting a set of one or more antibodies separately or as combination and singly or repeatedly into a primate;
b) obtaining a biological fluid or tissue sample;
c) analyzing the response against the variable heavy and light chain domains to identify the regions recognized by the idiotypic response;
d) using this information to design a de-immunized variant of the original antibody or a variant exhibiting reduced immunogenicity.

11. The method of claim 10, wherein at least one antibody has been produced in plant cells and at least one antibody has been produced in cells other than plant.

12. The method of claim 10, further comprising the steps of binding an anti-idiotypic antibody in primate sera or plasma to a specific idiotype or clonally related idiotypes or idiotypes derived by generating variants or mutants and visualized using an antibody that does not bind to the constant domains of the antibodies that have been injected into the primate.

13. A method of assessing the change in neutralization activity of an HIV antibody due to the presence of anti-idiotypic antibodies comprising the steps of:
a) determining a suitable concentration range of a neutralizing antibody in an appropriate biological assay, wherein the observed neutralization is in the range of 10 to 90%, preferably 20 to 85%, more preferably between 25 to 80% and most preferably between 50 to 80%.
b) mixing the neutralizing antibody with a mammalian diluted sera containing anti-idiotypic antibodies, such that the final concentration of the neutralizing antibody lies within the range determined in the previous step
c) incubating the mixture
d) determining the change in neutralization activity.

14. The method of claim 13 wherein the HIV antibody is an antibody variant having reduced or low immunogenicity.

15. A method of treatment for preventing, controlling or managing an infectious disease, comprising the administration of a set of one or more plant-derived human monoclonal antibodies either singly or as a cocktail wherein the total administered is selected from the group consisting of: 30-50 mg/kg, 10-40 mg/kg, 5-10 mg/kg and 0.1-5 mg/kg.

16. The method of claim 15, wherein one or more of the set of monoclonal antibodies prevents HIV infection following prophylactic administration.

17. The method of claim 15, wherein one or more of the set of monoclonal antibodies prevents or controls HIV infection when administered post-infection.

18. The method of claim 15 wherein the administration is subcutaneous, intravenous, intraperitoneal, transdermal or oral.

19. The method of claim 15, wherein one or more of the set of monoclonal antibodies is intended to prevent mother-to-child-transmission of HIV.

20. The method of claim 19, wherein the antibodies are administered to the pregnant female prior to delivery and to the newborn at or post-delivery.

21. The method of claim 20, wherein the antibodies administered to the pregnant female are given intravenously and more preferably subcutaneously.

22. The method of claim 20, wherein the newborn or baby is given the set of one or more plant-derived antibodies subcutaneously or more preferably orally.

23. The method of claim 22, wherein the set of one or more plant-derived antibodies is given repeatedly to the baby.

24. The method of claim 19, wherein the antibodies are repeatedly given to the baby orally.

25. The method of claim 15, wherein a set of one or more plant-derived antibodies are given once or repeatedly to a newborn or baby born to a pregnant female who is undiagnosed or has not been treated with ARV for at least four months.

26. The method of claim 15 wherein the antibodies are administered to a pregnant female has not been treated with ARV for at least four months.

27. A method of treatment for preventing or controlling an infectious disease, wherein different immunogenic monoclonal antibodies are administered serially.

28. The method of claim 27, where one or more of the set of monoclonal antibodies prevents HIV infection following prophylactic administration.

29. The method of claim 27, where one or more of the set of monoclonal antibodies controls HIV infection when administered post infection.

30. The method of claim 27, wherein the administration is subcutaneous, intravenous, intraperitoneal, transdermal or oral.

31. The method of claim 27, wherein one or more of the set of monoclonal antibodies is intended to prevent mother-to-child-transmission of HIV.

32. A method of treatment as in claim 27, wherein a set of one or more plant-derived antibodies are administered to the pregnant female prior to delivery and to the newborn at or post-delivery.

33. The method of claim 32, wherein the set of one or more plant-derived antibodies administered to the pregnant female are given intravenously or subcutaneously.

34. The method of claim 32, wherein the newborn or baby is given the set of one or more plant-derived antibodies subcutaneously or orally.

35. The method of claim 34, wherein the set of one or more plant-derived antibodies is given repeatedly to the newborn or baby.

36. The method of claim 34, wherein the antibodies are repeatedly given to the newborn or baby orally.

37. The method of claim 27, wherein the given the set of one or more plant-derived antibodies are given to a newborn or baby born to an untreated, treated for less than 4 months, or undiagnosed mother once or repeatedly.

38. The method of claim 27 wherein the monoclonal antibody is administered at a dose of 0.1 to 5 mg/kg pre-exposure, pre-infection or prophylactically.

39. The method of claim 31 wherein the monoclonal antibody is administered at a dose of 5 to 40 mg/kg for post-exposure treatment or for controlling or managing the infectious disease.

40. The method of claim 31 wherein the antibodies are administered to a pregnant female has not been treated with ARV for at least four months.

41. A method of treatment for preventing or controlling an infectious disease, comprising the administration of at least one antibody that has been produced in plant cells and at least one antibody has been produced in cells other than plant.
